(19) United States
(12) Patent Application Publication
(10) Pub. No.: US 2003/0147914 A1
(43) Pub. Date: Aug. 7, 2003

(54) Mycoplasma bovis vaccine and methods of reducing pneumonia in animals

(75) Inventors: Robin Lee Keich, Waterford, CT (US); David Ross McGavin, East Lyme, CT (US); Robert J. Yancey, Salem, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/177,857

(22) Filed: Jun. 20, 2002

Related U.S. Application Data
(60) Provisional application No. 60/302,638, filed on Jul. 2, 2001.

(51) Int. Cl. A61K 39/295; A61K 39/116; A61K 39/04

(52) U.S. Cl. 424/201.1; 424/203.1; 424/248.1

(57) ABSTRACT

The present invention relates to Mycoplasma bovis vaccines and methods for treating or preventing a disease or disorder in an animal caused by infection by Mycoplasma bovis by administering to the animal an effective amount of a Mycoplasma bovis vaccine. The Mycoplasma bovis vaccine can be a whole or partial cell inactivated or modified live preparation, a subunit vaccine, or a nucleic acid or DNA vaccine. The Mycoplasma bovis vaccine administered in accordance with the present can be synthesized or recombinantly produced. The invention also relates to combination vaccines, methods of preparing Mycoplasma bovis vaccines and kits.

### FIELD OF THE INVENTION

This invention relates to Mycoplasma bovis vaccine formulations and methods for treating or preventing a disease or disorder in an animal caused by infection by Mycoplasma bovis. The Mycoplasma bovis vaccine can be a whole or partial cell inactivated or modified live preparation, a subunit vaccine or a nucleic acid or DNA vaccine. The Mycoplasma bovis vaccine administered in accordance with the present invention can be synthesized or recombinantly produced.

### BACKGROUND OF THE INVENTION

Mycoplasma bovis is an important global bovine pathogen in housed or intensively reared beef and dairy cattle. The most frequently reported clinical manifestation is pneumonia of calves, which is often accompanied by arthritis, also known as pneumonia-arthritis syndrome. Its etiological role has also been associated with mastitis, otitis, and reproductive disease or disorders of cows and bulls. Significant economic losses are linked with M. bovis induced respiratory disease, since M. bovis has been associated with up to 36% of the mortality due to bovine respiratory disease (BRD). In order to reduce mortality, antibiotic therapy is often used since no fully licensed vaccines are currently available. Prevention of M. bovis disease may also reduce predisposition of the animal to other respiratory diseases. Therefore, a M. bovis bacterin that is highly efficacious and safe for young calves would be very valuable to the cattle industry.

### SUMMARY OF THE INVENTION

The present invention provides Mycoplasma bovis vaccines and methods of treating or preventing a disease or disorder caused by infection with Mycoplasma bovis by administering to an animal an effective amount of a Mycoplasma bovis vaccine and a pharmaceutically acceptable carrier. The vaccines of the present invention are provided in an amount sufficient to elicit or increase Mycoplasma bovis specific cellular or humoral primary and secondary immune responses. In one aspect, the animal is a calf. The present method of vaccination provides protection to calves against challenge with M. bovis. Furthermore, the present method of vaccination using a Mycoplasma bovis vaccine provides increased immunocompetence to calves and thereby increased resistance to other BRD pathogens, e.g., decreased predisposition to infection and disease caused by, but not limited to, but not limited to, bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica. The present method also provides Mycoplasma bovis vaccines and methods of eradicating Mycoplasma bovis from infected herds by administering to an animal an effective amount of a Mycoplasma bovis vaccine and a pharmaceutically acceptable carrier.

The Mycoplasma bovis vaccine administered in accordance with the present invention may include additional components, such as an adjuvant and optionally a second or more antigens for use in a combination vaccine. A second antigen is selected from the following, including but not limited to bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica.

The invention also provides a method for the preparation of a Mycoplasma bovis vaccine which comprises growing a isolate of Mycoplasma bovis in culture in a suitable medium; treating the Mycoplasma bovis with binary etheleneimine to inactivate the Mycoplasma bovis, and admixing the, inactivated Mycoplasma bovis with a suitable pharmaceutically acceptable carrier so as to formulate a bacterin.

The present invention further provides kits comprising Mycoplasma bovis and an adjuvant and optionally an antigen selected from the following, including but not limited to, bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica.

### BRIEF DESCRIPTION OF THE DRAWINGS

FIG. 1 is a graph showing group mean body temperature immediately prior to and following experimental M. bovis challenge. Calves vaccinated with two doses of the M. bovis bacterin (Group A) had significantly lower mean body temperatures on days 4-8, days 10-18 and day 20 when compared to the placebo vaccinated animals (Group B).

FIG. 2 is a graph showing group mean body temperature immediately prior to and following experimental M. bovis challenge. Calves vaccinated with two doses of the M. bovis bacterin (Groups A, B and C) had significantly lower mean body temperatures on days 7-17 when compared to the placebo vaccinated animals (Group D).

FIG. 3 is a graph showing group mean body temperature immediately prior to and following experimental M. bovis challenge. Calves vaccinated with two doses of the M. bovis bacterin (Treatment Groups 2, 3, 4, and 5) had significantly lower mean body temperatures on days 5-20 when compared to the placebo vaccinated animals (Treatment Group 1).

### DETAILED DESCRIPTION OF THE INVENTION

The present invention encompasses a vaccine and method of treating or preventing a disease or disorder in an animal caused by infection with Mycoplasma bovis comprising administering to the animal an effective amount of an inactivated Mycoplasma bovis vaccine and a pharmaceutically acceptable carrier. The invention encompasses methods of preparing M. bovis vaccines and M. bovis vaccine kits. Examples of Mycoplasma bovis strains are ATCC 25025 (deposited by R. G. Wittler on Oct. 8, 1968), 25523 (deposited by R. G. Wittier on Oct. 22, 1969) and 27368 (deposited by R. G. Wittler on Jul. 5, 1972), all of which deposits were made with the American Type Culture Collection, 1801 University Boulevard, Manassas, Va. 20110-2209. In a preferred embodiment, the Mycoplasma bovis isolate of the bacterin comprises one or more of the following strains: 2300, 3625, 16150, 20518 or 5063.

The present invention contemplates that any inactivated Mycoplasma bovis isolate may be formulated into an effective bacterin. In a preferred embodiment, the Mycoplasma bovis isolates inactivated with binary ethyleneimine (BEI), may be formulated into an effective bacterin. A deposit of the Mycoplasma bovis isolate strains 2300, 3625, 16150, 20518 or 5063 was made pursuant to the Budapest Treaty on the International Recognition of the Deposit of Microorganisms for the Purpose of Patent Procedure, with the American Type Culture Collection, 10801 University Boulevard, Manassas, Va. 20110-2209, and designated as strains PTA-3558, -3559, -3560, -3561 and -3685, respectively.

In certain embodiments, the vaccines used in the method of the present invention comprise a partial or whole cell M. bovis inactivated preparation (bacterin) or modified live vaccine and a pharmaceutically acceptable carrier, or partial or whole cell M. bovis inactivated preparation (bacterin) or modified live vaccine and an adjuvant.

For clarity of disclosure, and not by way of limitation, the detailed description of the invention is divided into the following subsections which describe or illustrate certain features, embodiments or applications of the invention.

### Definitions and Abbreviations

The abbreviation M., preceding the name of a species, refers to the genus Mycoplasma.

The term “treating or preventing” with respect to a Mycoplasma bovis infection as used herein means to inhibit the replication of Mycoplasma bovis bacteria, to inhibit Mycoplasma bovis shedding or transmission, or to prevent Mycoplasma bovis from establishing itself in its host, and to alleviate the symptoms of the diseases or disorders caused by Mycoplasma bovis infection or to accelerate the clearance of M. bovis from the animal. The treatment is considered therapeutic if there is a reduction in bacterial load, decrease in pulmonary infections, reduction in lung lesions, reduced rectal temperatures and/or increase in weight gain and/or growth. The method of the present invention is, for example, effective in preventing or reducing pneumonia, respiratory infections and lung lesions, reducing the level of M. bovis in the lung, reducing temperatures, and increasing weight gains in animals and especially cattle.

The term “M. bovis vaccine” as used herein refers to a vaccine useful in prevention or treating a disorder or disease caused by infection by M. bovis. M. bovis vaccine can include any vaccine effective in treating or preventing infection in cattle by virulent M. bovis. The M. bovis vaccine that may be used in the present invention can include, for example, a whole or partial M. bovis cell preparation, inactivated or modified live vaccines, a subunit vaccine having one or more M. bovis derived polypeptides or proteins, or immunogenic fragments of such proteins or polypeptides, or one or more M. bovis genes or nucleic acids encoding for one or more M. bovis derived polypeptides or proteins, or immunogenic fragments thereof, and which genes or nucleic acids are capable of being expressed in vivo in cattle. The M. bovis polypeptides, proteins, immunogenic fragments of such polypeptides and proteins, or M. bovis genes or nucleic acids can be synthesized or recombinantly produced using techniques known in the art. Preferably, the M. bovis vaccine used in the method of the present invention is a bacterin.

The term immunogenic fragment as used herein refers to a fragment of a protein from M. bovis, which is capable of inducing an immune response in a host animal. The immune response may comprise, without limitation, induction of cellular and/or humoral immunity.

The term “animal” as used herein refers to all non-human animals, including mammals.

The term “cattle” as used herein refers to bovine animals including but not limited to steer, bulls, cows, and calves. Preferably, the method of the present invention is applied to an animal which is a non-human mammal; most preferably, a calf.

The term “bacterin” as used herein refers to a preparation of inactivated whole or partial M. bovis cells suitable for use as a vaccine.

The term “immunologically effective amount” refers to an amount of M. bovis vaccine sufficient to elicit an immune response in the subject to which it is administered. The immune response may comprise, without limitation, induction of cellular and/or humoral immunity. An effective amount of M. bovis vaccine means, for example, that the bacterin prevents or reduces the severity of mycoplasmal pneumonia.

The term “adjuvant” as used herein, is a potentiator of the immune response.

The term “pharmaceutically acceptable carrier” refers to a carrier medium that does not interfere with the effectiveness of the biological activity of the active ingredient, is chemically inert and is not toxic to the subject to whom it is administered.

### Inactivated (Partial or Whole Cell) and Modified Live Vaccines

The invention provides a Mycoplasma bovis vaccine and a method for the preparation of a Mycoplasma bovis vaccine which comprises growing a isolate of Mycoplasma bovis in culture in a suitable medium; treating the Mycoplasma bovis with binary ethyleneimine to inactivate the Mycoplasma bovis, and admixing the inactivated Mycoplasma bovis with a suitable pharmaceutically acceptable carrier so as to formulate a bacterin. In one embodiment Mycoplasma bovis is isolated from lung tissue. In another embodiment, Mycoplasma bovis is isolated from lymph node tissue. A variety of such carriers are well known in the art and include distilled or deionized water, saline, or mineral oil. In addition to inactivated bacterial isolates, a bacterin product can also include an appropriate amount of one or more commonly used adjuvants. Suitable adjuvants may include, but are not limited to: mineral gels, e.g., aluminum hydroxide; surface active substances such as lysolecithin; glycosides, e.g., saponin and saponin derivatives such as Quil A or GPI-0100; cationic surfactants, e.g. DDA (quaternary hydrocarbon ammonium halogenides, pluronic polyols; polyanions and polyatomic ions; polyacrylic acids, non-ionic block polymers, e.g., Pluronic F-127 (B.A.S.F., USA); Avridine and Rantidine; peptides; recombinant mutant labile toxins, e.g., leukotoxin (LT) or cholera toxin (CT); chemically bound or close proximity molecular transporters; mineral oils, e.g. Montanide ISA-50 (Seppic, Paris, France), carbopol, Amphigen (Hydronics, USA), Omaha, Nebr. USA, Alhydrogel, (Superfos Biosector, Frederikssund, Denmark) oil emulsions, e.g. an emulsion of mineral oil such as BayolF/Arlacel A and water, or an emulsion of vegetable oil, water and an emulsifier such as lecithin; alum, cholesterol cytokines and combinations of adjuvants. Polyatomic ions can also function as dispersing, thickening and anticaking agents which allow the vaccine to be resuspended as a mondisperse suspension after a prolonger period of settling. The adjuvant combinations may be presented in aqueous, encapsulated (controlled or delayed release) or microencapsulated forms. The immunogen may also be incorporated into liposomes, or conjugated to polysaccharides and/or other polymers for use in a vaccine formulation. Additional substances that can be included in a bacterin product for use in the present methods include, e.g., one or more preservatives such as disodium or tetrasodium salt of Ethylene-Diamine Tetra Acetic acid (EDTA), merthiolate, and the like. Vaccines are formulated as liquid dosage or presented in a solid dosage with the making up a soluble component or a microparticulate that is resuspended in a pharmaceutically acceptable diluent prior to use. Methods of preparing soluble components or microparticulates include, but are not limited to, biacervation, congelgation, spray drying, bubble srying, precipitation, supercritical sovlation/encapsulation and lyophilization. In a preferred embodiment, the Mycoplasma bovis isolate designated 2300 is used in formulating the bacterin. In a further preferred embodiment, the adjuvant combination of Quil A, Amphigen, and cholesterol is used in formulating the bacterin.

The precise conditions under which the isolate is grown may vary depending upon the precise composition of the medium and the specific isolate being grown. However the isolate is typically grown from about 24 hours to about 72 hours, measured from the time of incubation to the time of harvest. The virulent Mycoplasma bovis isolate so grown is then treated with binary ethyleneimine (BEI) to inactivate the Mycoplasma bovis as described in U.S. Pat. No. 5,565,205, or inactivated with formalin, glutaraldehyde, heat, irradiation, BPL or other inactivants known to the art. For example, where the isolate is treated with BEI, the culture of the isolate may be contacted with BEI at a concentration of about 2 to about 10 mM. The culture is then incubated under conditions effective to inactivate Mycoplasma bovis e.g., for at least about 24 hours at about 37degrees C. The BEI culture is then neutralized by adding sodium thiosulfate at an effective neutralizing concentration, e.g. 2 to 10 mM.

The resulting, inactivated Mycoplasma bovis may be concentrated. Various methods are known in the art for concentrating such organisms. For example, the organisms may be concentrated by centrifugation, e.g. ultracentrifugation, or by filtration, e.g. ultrafiltration.

The concentrated, inactivated Mycoplasma bovis which result are then recovered, using methods well known in the art. Finally, the resulting concentrated, inactivated Mycoplasma bovis so recovered is admixed with a suitable pharmaceutically acceptable carrier so as to formulate the bacterin. The bacterin may also be produced by any of several modifications to the preceding method, which are readily known to the skilled artisan.

M. bovis isolates can also be obtained directly from infected cattle lung lesions using known techniques. M. bovis isolates can also be obtained directly from infected cattle lymph node tissue using known techniques. M. bovis isolates can also be obtained directly from infected cattle lymph node tissue using known techniques. The present invention also contemplates preparation of modified live M. bovis vaccines, such as by attenuation of virulent strains by passage, which technique is known in the art.

Suitable preparations of the vaccines of the present invention include injectables, either as liquid solutions or suspensions; solid forms suitable for solution in, or suspension in, liquid prior to injection, may also be prepared. The preparation may also be emulsified.

Inactivated Mycoplasma bovis isolates can also be combined with the following bacteria and viruses, including but not limited to, bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica.

### Subunit Vaccines

The method of the present invention can be practiced using subunit vaccines having purified M. bovis immunogenic proteins, polypeptides and immunogenic fragments of such proteins and polypeptides. Such proteins and polypeptides can be prepared using techniques known in the art, for example extracts prepared using surface action agents, or thermal, chemical and mechanical extracts. Further, methods which are well known to those skilled in the art can be used to determine protein purity or homogeneity, such as polyacrylamide gel electrophoresis of a sample, followed by visualizing a single polypeptide band on a staining gel. Higher resolution may be determined using HPLC or other similar methods well known in the art.

In a specific embodiment, the vaccine used in the present invention comprises at least one protein of M. bovis such as, but not limited to P13, P18, P21, P25-26, P33-34, P39-40, P45-46, P50, P54-58, P77, P82, P87-89 P97, and P175.

In other embodiments the subunit vaccine of the present invention comprises at least one other immunogenic or antigenic molecule which is not a M. bovis protein, polypeptide or immunogenic fragment thereof and is preferably a viral or bacterial antigen. In a preferred embodiment the antigen is bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, or Manheimia haemolytica. Such a composition is beneficial as a combination vaccine. The subunit vaccines and combination vaccines of the present invention can be employed in the methods of the present invention to treat or prevent diseases or disorders caused by M. bovis infection.

In a further specific embodiment, the immunogenic fragments of such proteins or polypeptides have a sequence comprising at least 10, at least 20, at least 30, at least 40, at least 50 or at least 100 contiguous amino acids of the immunogenic proteins and polypeptides used in the method of the present invention, including but not limited to P13, P18, P21, P25-26, P33-34, P39-40, P45-46, P50, P54-58, P77, P82, P87-89 P97, and P175.

Further, the M. bovis proteins for use in vaccines are substantially pure or homogeneous. The method of the present invention uses proteins or polypeptides which are typically purified from host cells expressing recombinant nucleotide sequences encoding these proteins. Such protein purification can be accomplished by a variety of methods well known in the art. See, for example, the techniques described in “Methods In Enzymology”, 1990, Academic Press, Inc., San Diego, “Protein Purification: Principles and practice”, 1982, Springer-Verlag, New York.

Purified M. bovis polypeptides and proteins and immunogenic fragments thereof can also be prepared using known synthetic methods.

M. bovis polypeptides and proteins and immunogenic fragments thereof can also be expressed and delivered using live recombinant viral and bacterial vectors such as adenovirus or Salmonella. The actual vectors are also known and readily available within the art or can be constructed by one skilled in the art using well-known methodology.

### Gene and Nucleic Acid Vaccines

The method of the present invention can be practiced using M. bovis genes or nucleic acids encoding for immunogenic proteins, polypeptides and immunogenic fragments of such proteins and polypeptides. Such genes and nucleic acids can be expressed in vivo and can be prepared using techniques known in the art.

In a specific embodiment, the vaccine used in the present invention comprises at least one gene or nucleic acid encoding for a protein of M. bovis such as, but not limited to, P13, P18, P21, P25-26, P33-34, P39-40, P45-46, P50, P54-58, P77, P82, P87-89 P97, and P175.

In a further specific embodiment, the genes or nucleic acids used in the method of the present invention encode for the immunogenic fragments of the M. bovis proteins or polypeptides and have a sequence comprising at least 10, at least 20, at least 30, at least 40, at least 50 or at least 100 contiguous amino acids of the immunogenic proteins and polypeptides used in the method of the present invention, including but not limited to P13, P18, P21, P25-26, P33-34, P39-40, P45-46, P50, P54-58, P77, P82, P87-89 P97, and P175.

In other embodiments of the method of the present invention, the gene or nucleic acids used are administered by known methods, such as, for example, by use of a gene gun or other needle-free delivery devices.

In yet other embodiments of the method of the present invention, the gene or nucleic acids used are DNA vaccines. Further, the nucleic acid or genes can be present in association with liposomes or other transfection facilitating agents, as are known in the art.

Methods for the preparation and delivery of DNA vaccines are known in the art. See, for example, Krishnan, B. R, “Current Status of DNA vaccines in veterinary medicine”, Advanced Drug Delivery Reviews, Elsevier Science (2000)

### Dosing, Modes of Administration and Treatment

According to the present invention, at least one dose of an effective amount of a M. bovis vaccine administered to an animal and preferably a calf of approximately one to tens weeks of age provides effective immunity against a later challenge of M. bovis. Preferably, the M. bovis vaccine is administered at about 7 to 28 and again at about 28 to 48 days of age. The effective amount of a M. bovis bacterin vaccine contains about 1×10⁶ to about 5×10¹⁰ colony forming units (CFU) per dose. Preferably, a M. bovis bacterin vaccine that provides effective immunity contains about 1×10⁸ to about 5×10¹⁰ CFU/dose and more preferably, about 5×10⁸ to about 5×10¹⁰ CFU/dose.

According to the present invention, the effective amount of M. bovis bacterin vaccine for administration is about 0.5 to about 5.0 ml, preferably about 1.5 ml to about 2.5 ml, and more preferably, about 2 ml.

The amount of a M. bovis vaccine which is a subunit vaccine comprising one or more proteins or polypeptides or immunogenic fragments of such proteins or polypeptides effective in the method of the present invention is from about 0.01 μg to about 200 μg.

The amount of a M. bovis vaccine which is a vaccine comprising one or more M. bovis genes or nucleic acids (preferably DNA) encoding for immunogenic proteins or polypeptides or immunogenic fragments of such proteins or polypeptides effective in the method of the present invention is from about 0.1μg to about 200 mg. In accordance with the present invention, administration can be achieved by known routes, including the oral, intranasal, mucosal topical, transdermal, and parenteral (e.g., intravenous, intraperitoneal, intradermal, subcutaneous or intramuscular). Administration can also be achieved using needle-free delivery devices. Administration can be achieved using a combination of routes, e.g., first administration using a parental route and subsequent administration using a mucosal route. A preferred route of administration is subcutaneous or intramuscular administration.

The present invention also contemplates a single dose vaccination method, which eliminates the necessity of administration of additional doses to calves in order to generate and/or maintain immunity against M. bovis.

According to the present invention, the administration of an effective amount of a Mycoplasma bovis bacterin administered to calves at approximately three and six weeks of age provides effective immunity against respiratory infections, including pneumonia, reduces lung lesions, reduces the level of M. bovis in the lung, reduces temperatures, and increases weight gains.

The present invention provides a method of immunizing a calf against infection by Mycoplasma bovis comprising administering to the calf at least one dose, and preferably two doses of the bacterin so as to immunize the calf against Mycoplasma bovis infection. In a preferred embodiment, the bacterin is administered subcutaneously. Moreover, it is preferred that the bacterin dose comprise about 2 ml of the bacterin, each ml containing about 2.5×10⁸ Mycoplasma bovis colony forming units. The bacterin is desirably administered twice to the calf; once at about three weeks, and once at about six weeks, after the birth of the calf.

The present invention also contemplates that the administration of an effective amount of a Mycoplasma bovis bacterin administered to animals, and preferably cattle to treat or prevent disorders including pneumonia, arthritis, mastitis, otitis and reproductive disorders in such animals.

### Vaccine Kits

The invention also provides a pharmaceutical kit comprising one or more containers comprising one or more of the ingredients of the vaccine formulations of the invention. The present invention thus provides a method of immunizing an animal, or treating or preventing various diseases or disorders in an animal, comprising administering to the animal an effective immunizing dose of a vaccine of the present invention. In a preferred embodiment the kit comprises in a container a inactivated Mycoplasma bovis isolate and an adjuvant selected from Quil A or GPI-0100, DDA, saponin, cholesterol, aluminum gel, carbopol, Amphigen, Alhydrogel, oil in water, water in oil, cytokines, or combinations of adjuvants. In another embodiment, the kit of the present invention optionally comprises, in the same container or in a second container, antigens selected from the following, including but not limited to bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, or Manheimia haemolytica.

### Packaging

The vaccine compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

The present invention is further illustrated by the following examples.

### EXAMPLE 1

### Materials and Methods

Animals

Healthy crossbred dairy calves at approximately fourteen days of age were obtained for vaccination. Calves were acclimatized for seven days prior to the initiation of the study. All calves received a concentrated non-medicated diet daily, free of any known contaminants or pesticides and had free access to water.

Vaccines

The bacterins contained a BEI inactivated whole cell M. bovis bacteria at an appropriate concentration per dose. In addition, each vaccine preparation contained phosphate buffered saline (PBS) and an appropriate adjuvant. The placebo contained either PBS or PBS and an oil in water adjuvant.

Challenge Method

Each calf received either 10 or 12 ml of a fresh M. bovis culture [approximately 1×10⁸ to 1×10¹⁰ colony forming units (CFU/ml)] by the intranasal route on three consecutive days. A viable count (CFU/ml) of the challenge inoculum was determined shortly after the completion of each experimental challenge.

Experimental Procedure

A unique ear tag number identified each calf. Animals were randomly assigned by age into pens and treatment groups.

Animals were vaccinated with 2 ml of the appropriate vaccine or placebo by the subcutaneous route on day 0 (left neck) and on day 21 (right neck).

All animals were weighed at 1 day prior to challenge, 7 days following challenge, 14 days following challenge, and at approximately 3 weeks following challenge.

Rectal temperatures were measured each morning 1-day prior to challenge, immediately prior to challenge, and for 20 days following challenge.

A blood sample was collected from each calf from the jugular vein. Calves were bled at approximately 1 day prior to first vaccination, 1 day prior to second vaccination, 1 day prior to challenge (approximately 3 weeks post-second vaccination), 7 days following challenge, 14 days following challenge, and at necropsy (approximately 3 weeks post-challenge). Serum from each blood sample was stored at −20° C. until evaluated by a M. bovis ELISA kit (Chekit M. bovis Sero) prepared by Bommeli AG (Hoechst Roussel Vet Diagnostics, Liebefeld-Bern, Switzerland). The ELISA plates were read using a Multiscan reader at a wavelength of 405 nm. Optical density (OD) values were translated to a percentage relating to the OD value of the positive control serum, using the following formula: percentage=(Sample OD-Negative serum OD)/(positive serum OD-Negative serum OD)*100. Values lower than 60% were considered negative. Sera having percentages between 60 and 80% were considered suspect, while sera showing OD greater than 80% were accepted as positive.

All animals were necropsied at approximately 3 weeks following the experimental M. bovis challenge. Calves were euthanized and all major organs, excluding the central nervous system, were examined grossly.

Lungs were removed and evaluated grossly for characteristic lesions attributable to a M. bovis infection. Lesions were sketched on a standard lung diagram. Percent gross involvement per each lung lobe was weighted using the following ratios of individual lung lobes to total lung mass.

| Lung Lobe | Percentage of Lung |
|---|---|
| Left Apical | 5 |
| Right Apical | 6 |
| Middle | 5 |
| Left Cardiac | 6 |
| Right Cardiac | 7 |
| Accessory | 4 |
| Left Diaphragmatic | 32 |
| Right Diaphragmatic | 35 |

The weighted lung lobe values were then summed in order to determine the percentage of total lung with gross lesions (Pointon et al, 1992). In addition the following formula was used to calculate the percent reduction.

$$100 - \frac{\text{Mean Percent Lung Damage of Treatment Group}}{\text{Mean Percent Lung Damage of Control Group}} = \text{Percent Reduction}$$

In addition, each lung was lavaged with 50 ml of PBS. Attempts were made to isolate and determine the viable M. bovis counts from the bronchial lavage fluid. The M. bovis viable count (CFU/ml) was determined by preparing appropriate serial dilutions of bronchial lavage fluid and plating samples onto an appropriate agar medium.

### EXAMPLE 2

In this example, the efficacy of a M. bovis bacterin was evaluated in young calves. Twenty-four, healthy crossbred calves, were randomly assigned by age.

Animals were vaccinated with 2 ml of either the vaccine or placebo by the subcutaneous route on day 0 (left neck) and on day 21 (right neck). The experimental treatment groups and vaccines used are shown in Table 1.

TABLE 1
Experimental Treatment Groups

| Treatment | | |
|---|---|---|
| Group | Experimental Vaccines (2 ml dose) | Number of Animals |
| A | M.bovis (5 × 10⁸ CPU) + Amphigen | 11 |
| B | Placebo (PBS + Amphigen) | 13 |

Calves were challenged as described above at 3 weeks following second vaccination. Each calf received 10 ml of a fresh M. bovis culture by the intranasal route on three consecutive days.

A viable count (CFU/ml) of each challenge inoculum was determined within one hour after the completion of the M. bovis experimental challenge. Results are shown in Table 2.

TABLE 2
Viable Count (CFU/ml) of Mycoplasma bovis Challenge Inoculum

| Challenge Culture | CFU/ml |
|---|---|
| Day 1 | 5.0 × 10⁹ |
| Day 2 | 1.0 × 10⁹ |
| Day 3 | 1.2 × 10⁹ |

All animals were weighed at 1 day prior to challenge, 7 days following challenge, 14 days following challenge, and approximately 3 weeks following experimental M. bovis challenge. Results are summarized in Table 3. Calves that were administered the experimental M. bovis bacterin (Treatment Group A) had increased weight gains when compared to the placebo vaccinated group (Treatment Group B).

TABLE 3
Summary of Body Weights Following Experimental Mycoplasma bovis Challenge
Mean Body Weight (kg) ± Standard Deviation

| | | 1 Week | 2 Weeks | 3 Weeks | |
|---|---|---|---|---|---|
| Treatment | Prior to | Post- | Post- | Post- | |
| Group | Challenge | Challenge | Challenge | Challenge | Weight Gain |
| A | 94.8 ± 12.9 | 98.7 ± 13.9 | 107.3 ± 13.6 | 114.6 ± 12.9 | 19.8 |
| B | 104.0 ± 15.6 | 106.8 ± 14.7 | 109.9 ± 14.1 | 113.0 ± 14.7 | 9.0 |

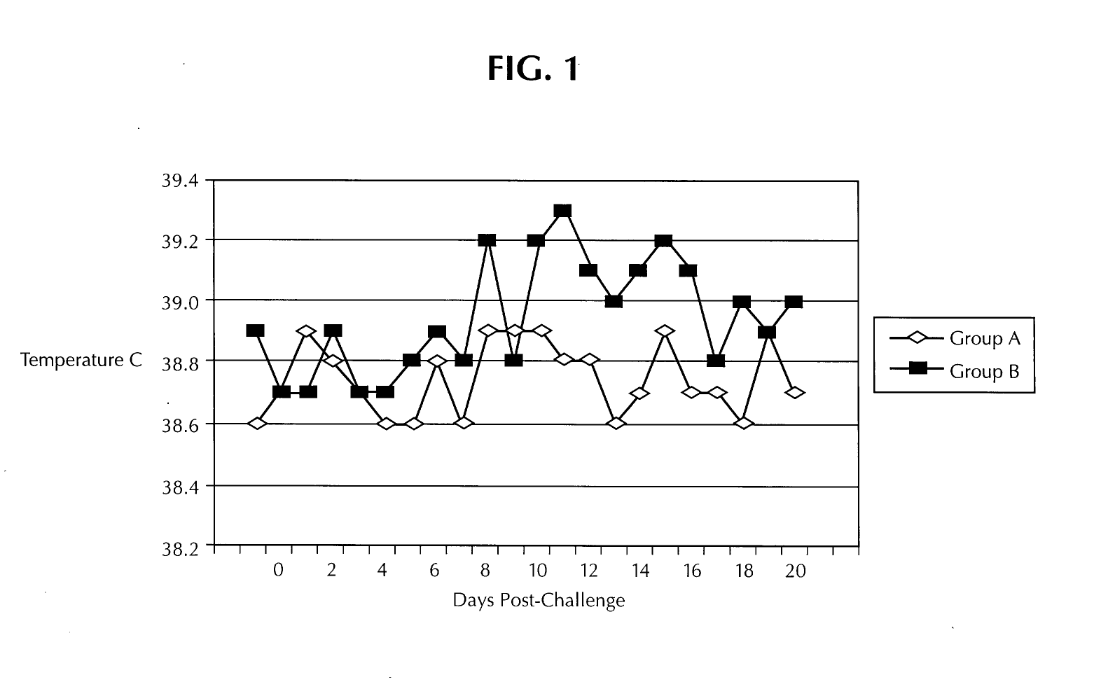

Rectal temperatures were measured each morning 1-day prior to challenge, immediately prior to challenge, and for 20 days following experimental M. bovis challenge. Results are summarized in FIG. 1. Calves vaccinated with the M. bovis bacterin (Treatment Group A) had lower mean body temperatures on days 4 through 8, days 10 through 18 and day 20 when compared to the placebo vaccinated animals (Treatment Group B).

M. bovis specific serum antibody responses (IgG) are summarized in Table 4. Serum samples with mean percentage optical density (OD) values>80% of the positive control serum were considered positive for M. bovis. All calves were M. bovis negative prior to vaccination. Calves that received the experimental M. bovis bacterin (Treatment Group A) were seropositive to M. bovis prior to second vaccination and remained seropositive throughout the study. Animals in Treatment Group B (placebo vaccinated animals) were seronegative until 2 weeks following the experimental M. bovis challenge.

TABLE 4
Summary of Mycoplasma bovis Serum Antibody (IgG) Mean Percentage of Optical
Density Values to Positive Control Serum ± Standard Deviation

| | | Prior to | | 1 Week | 2 Weeks | 3 Weeks |
|---|---|---|---|---|---|---|
| Treatment | Pre- | Second | Prior to | Post- | Post- | Post- |
| Group | Vaccination | Vaccination | Challenge | Challenge | Challenge | Challenge |
| A | 26.4 ± 29.1 | 210.2 ± 79.5 | 94.6 | 342.6 ± 12.6 | 392.5 ± 11.3 | 385.4 ± 13.2 |
| B | 29.9 ± 39.5 | 71.4 ± 64.8 | 24.9 ± 42.2 | 77.5 ± 55.5 | 250.7 79.7 | 326.6 ± 50.0 |

All animals were necropsied at approximately 3 weeks following the experimental M. bovis challenge. Lungs were removed and evaluated grossly for characteristic lesions attributable to a M. bovis infection. Percent lung damage scores and percent reduction of lung lesions are summarized in Table 5. Calves that were administered the experimental M. bovis bacterin (Treatment Group A) had a 71.2 percent reduction in lung damage scores when compared to the placebo vaccinated animals (Treatment Group B). These results demonstrate that two doses of the experimental M. bovis bacterin was capable of inducing protection in calves following experimental challenge.

TABLE 5
Summary of Percent Lung Damage Scores Mean Weighted
Percentage ± Standard Deviation

| Treatment | | |
|---|---|---|
| Group | Percent Lung Damage | Percent Reduction |
| A | 1.80 ± 3.04 | 71.2 |
| B | 6.25 ± 6.73 | — |

Each lung was lavaged with 50 ml of PBS. Results of the isolation of M. bovis from bronchial lavage samples approximately twenty-one days following the experimental M. bovis challenge are summarized in Table 6. Calves that were administered the experimental M. bovis bacterin (Treatment Group A) had a reduced incidence and level of viable M. bovis in lung lavage samples when compared to the placebo vaccinated calves (Treatment Group B).

TABLE 6
Summary of Mycoplasma bovis Isolations
from Lung Lavage Fluid

| Treatment Group | Number of Animals M.bovis Positive | CFU/ml |
|---|---|---|
| A | 3/11 | 3.27 × 10² |
| B | 13/13 | 2.41 × 10⁶ |

In conclusion, calves receiving the experimental M. bovis bacterin (Treatment Group A) developed less lung lesions, had reduced rectal temperatures, increased weight gain, and an approximately 4 log reduction in the level of viable M. bovis isolated from lung lavage samples when compared to the placebo administered animals (Treatment Group B). The results show that two doses of the M. bovis bacterin was capable of inducing a serological response and protection from a M. bovis experimental challenge.

### EXAMPLE 3

In this example, the efficacy of various M. bovis bacterins was evaluated in young calves. Fifty-eight, healthy crossbred calves, were randomly assigned by age.

Animals were vaccinated with 2 ml of the appropriate vaccine or placebo by the subcutaneous route on day 0 (left neck) and on day 21 (right neck). The experimental treatment groups and vaccines used are shown in Table 1.

TABLE 1
Experimental Treatment Groups

| Treatment | | Number of |
|---|---|---|
| Group | Experimental Vaccines (2 ml dose) | Animals |
| A | M. bovis (5 × 10⁸ CFU) + | 14 |
| | Amphigen + Alhydrogel | |
| B | M. bovis (5 × 10⁸ CFU) + | 14 |
| | Amphigen + QuilA/Cholesterol | |
| C | M. bovis (5 × 10⁸ CFU) + | 15 |
| | Amphigen | |
| D | Placebo (PBS) | 15 |

Calves were challenged as described above at 3 weeks following second vaccination. Each calf received 12 ml of a fresh M. bovis culture by the intranasal route on three consecutive days.

A viable count (CFU/ml) of each challenge inoculum was determined within one hour after the completion of the M. bovis experimental challenge. Results are shown in Table 2.

TABLE 2
Viable Count (CFU/ml) of Mycoplasma bovis Challenge Inoculum

| Challenge Culture | CFU/ml |
|---|---|
| Day 1 | 2.2 × 10⁹ |
| Day 2 | 3.2 × 10⁹ |
| Day 3 | 1.7 × 10⁹ |

All animals were weighed at 1 day prior to challenge, 7 days following challenge, 14 days following challenge, and approximately 3 weeks following experimental M. bovis challenge. Results are summarized in Table 3. Calves that were administered the experimental M. bovis bacterins (Treatment Groups A, B, and C) had increased weight gains when compared to the placebo vaccinated group (Treatment Group D).

TABLE 3
Summary of Body Weights Following Experimental Mycoplasma bovis Challenge
Mean Body Weight (kg) + Standard Deviation

| | | 1 Week | 2 Weeks | 3 Weeks | |
|---|---|---|---|---|---|
| Treatment | Prior to | Post- | Post- | Post- | Weight |
| Group | Challenge | Challenge | Challenge | Challenge | Gain |
| A | 79.79 ± 12.29 | 88.00 ± 13.86 | 98.43 ± 12.35 | 103.71 ± 10.76 | 23.92 ± 5.99 |
| B | 78.21 ± 9.50 | 86.93 ± 9.90 | 98.29 ± 8.47 | 105.21 ± 9.32 | 27.00 ± 5.23 |
| C | 78.07 ± 16.78 | 86.60 ± 17.11 | 98.00 ± 20.92 | 104.00 ± 21.56 | 25.93 ± 8.80 |
| D | 78.93 ± 19.16 | 88.60 ± 20.44 | 94.43 ± 20.01 | 96.93 ± 20.89 | 18.00 |

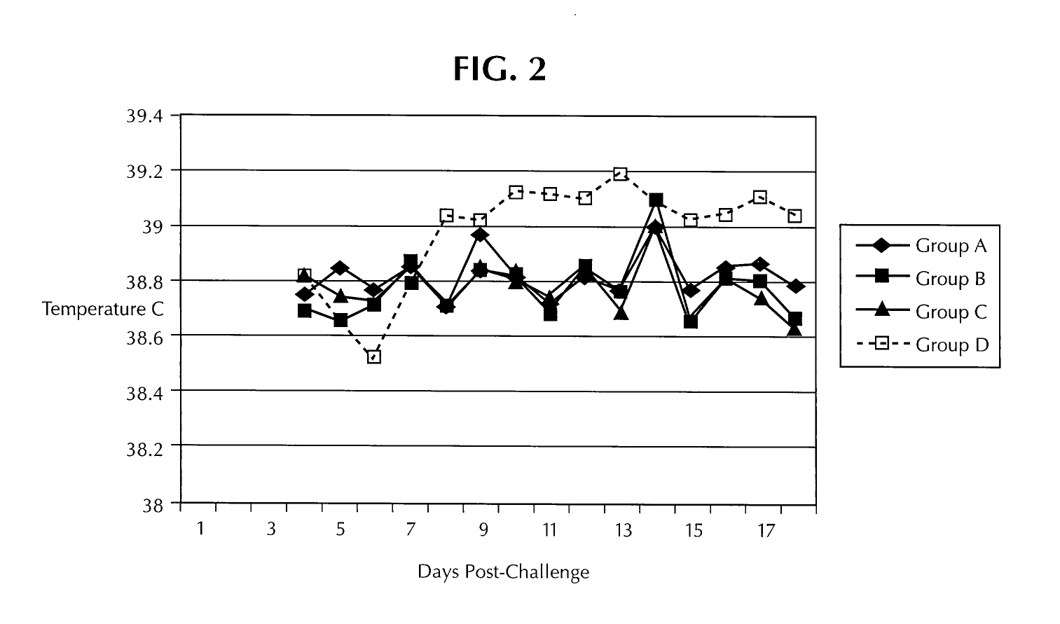

Rectal temperatures were measured each morning 1-day prior to challenge, immediately prior to challenge, and for 20 days following experimental M. bovis challenge. Results are summarized in FIG. 2. Calves administered two doses of the M. bovis vaccines (Treatment Groups A, B, and C) had lower mean body temperatures on days 7 through 17 when compared to the placebo vaccinated animals (Treatment Group D).

M. bovis specific serum antibody responses (IgG) are summarized in Table 4. Serum samples with mean percentage optical density (OD) values>80% of the positive control serum were considered positive for M. bovis. All calves were M. bovis negative prior to vaccination. Calves that received the experimental M. bovis bacterins (Treatment Groups A, B, and C) were seropositive to M. bovis prior to second vaccination and remained seropositive throughout the study. Animals in Treatment Group D (placebo vaccinated animals) were seronegative until 3 weeks following the experimental M. bovis challenge.

TABLE 4
Summary of Mycoplasma bovis Serum Antibody (IgG) Mean Percentage of Optical
Density Values to Positive Control Serum ± Standard Deviation

| | | Prior to | | 1 Week | 2 Weeks | 3 Weeks |
|---|---|---|---|---|---|---|
| Treatment | Pre- | Second | Prior to | Post- | Post- | Post- |
| Group | Vaccination | Vaccination | Challenge | Challenge | Challenge | Challenge |
| A | Negative | 244.3 ± 66.0 | 314.7 ± 10.5 | 134.9 ± 7.4 | 115.5 ± 8.0 | 142.5 ± 6.9 |
| B | Negative | 262.1 ± 86.9 | 309.9 ± 33.6 | 139.5 ± 7.5 | 114.9 ± 7.5 | 145.0 ± 4.1 |
| C | Negative | 184.5 ± 60.6 | 292.2 ± 93.7 | 141.1 ± 9.1 | 118.9 ± 7.5 | 140.4 ± 7.7 |
| D | Negative | 36.9 ± 70.6 | 37.2 ± 81.0 | 37.4 ± 27.9 | 53.2 ± 39.4 | 100.5 ± 99.6 |

All animals were necropsied at approximately 3 weeks following the experimental M. bovis challenge. Lungs were removed and evaluated grossly for characteristic lesions attributable to a M. bovis infection. Percent lung damage scores and percent reduction of lung lesions are summarized in Table 5. Calves that were administered the experimental M. bovis bacterins (Treatment Groups A, B, and C) had lower percent lung damage scores when compared to the placebo vaccinated animals (Treatment Group D). These results demonstrate that two doses of the experimental M. bovis bacterins were capable of inducing protection in calves following experimental challenge.

TABLE 5
Summary of Percent Lung Damage Scores Mean Weighted
Percentage ± Standard Deviation

| Treatment | | |
|---|---|---|
| Group | Percent Lung Damage | Percent Reduction |
| A | 1.71 ± 3.03 | 77.5 |
| B | 1.49 ± 3.23 | 80.4 |
| C | 3.61 ± 6.17 | 52.5 |
| D | 7.60 ± 15.93 | — |

Each lung was lavaged with 50 ml of PBS. Results of the isolation of M. bovis from bronchial lavage samples approximately twenty-one days following the experimental M. bovis challenge are summarized in Table 6. Calves that were administered the experimental M. bovis bacterins (Treatment Groups A, B, and C) had a reduced incidence and level of viable M. bovis in lung lavage samples when compared to the placebo vaccinated calves (Treatment Group D).

TABLE 6
Summary of Mycoplasma bovis Isolations
from Lung Lavage Fluid

| | Number of Animals | |
|---|---|---|
| Treatment Group | M. bovis Positive | CFU/ml |
| A | 5/14 | 1.93 × 10² |
| B | 1/14 | 42.9 |
| C | 9/15 | 1.34 × 10⁶ |
| D | 12/14 | 4.50 × 10⁶ |

In conclusion, calves receiving the experimental M. bovis bacterins (Treatment Groups A, B, and C) developed less lung lesions, had reduced rectal temperatures, increased weight gain, and a reduced level of viable M. bovis isolated from lung lavage samples when compared to the placebo administered animals (Treatment Group D). The results show that two doses of the M. bovis bacterins were capable of inducing a serological response and protection from a M. bovis experimental challenge.

### EXAMPLE 4

In this example, the efficacy of various M. bovis bacterin formulations was evaluated in young calves following either a homologous or heterologous challenge. Eighty-three, healthy crossbred calves, were randomly assigned by age.

Animals were vaccinated with 2 ml of the appropriate vaccine or placebo by the subcutaneous route on day 0 (left neck) and on day 21 (right neck). The experimental treatment groups and vaccines used are shown in Table 1.

TABLE 1
Experimental Treatment Groups

| Treatment | | Number of |
|---|---|---|
| Group | Experimental Vaccines (2 ml dose) | Animals |
| 1 | Placebo (PBS) | 16 |
| 2 | M. bovis strain 2300 (5 × 10⁸ CFU) + | 17 |
| | Amphigen + QuilA/Cholesterol | |
| 3 | M. bovis strain 3625 (5 × 10⁸ CFU) + | 16 |
| | Amphigen + GPI-0100/Cholesterol | |
| 4 | M. bovis strain 3625 (5 × 10⁸ CFU) + | 17 |
| | Amphigen + QuilA/Cholesterol | |
| 5 | M. bovis strain 5063 (5 × 10⁸ CFU) + | 17 |
| | Amphigen + QuilA/Cholesterol | |

Calves were challenged as described as described above at approximately 4 weeks following second vaccination. Each calf received 12 ml (6 ml per nostril) of a fresh M. bovis strain 5063 culture by the intranasal route on three consecutive days.

A viable count (CFU/ml) of each challenge inoculum was determined within one hour after the completion of the M. bovis experimental challenge.

All animals were weighed at 1 day prior to challenge and approximately 3 weeks following experimental M. bovis challenge. Results of the average daily weight gains are summarized in Table 2. Calves that were administered the experimental M. bovis bacterins (Treatment Groups 2, 3, 4, 5) had increased average daily weight gains when compared to the placebo vaccinated group (Treatment Group 1).

TABLE 2
Summary of Average Daily Weight Gains Following Experimental
Mycoplasma bovis Challenge Average Daily Weight Gain (kg)

| Treatment Group | Average Daily Weight Gain |
|---|---|
| 1 | 0.3 |
| 2 | 0.5 |
| 3 | 0.7 |
| 4 | 0.6 |
| 5 | 0.9 |

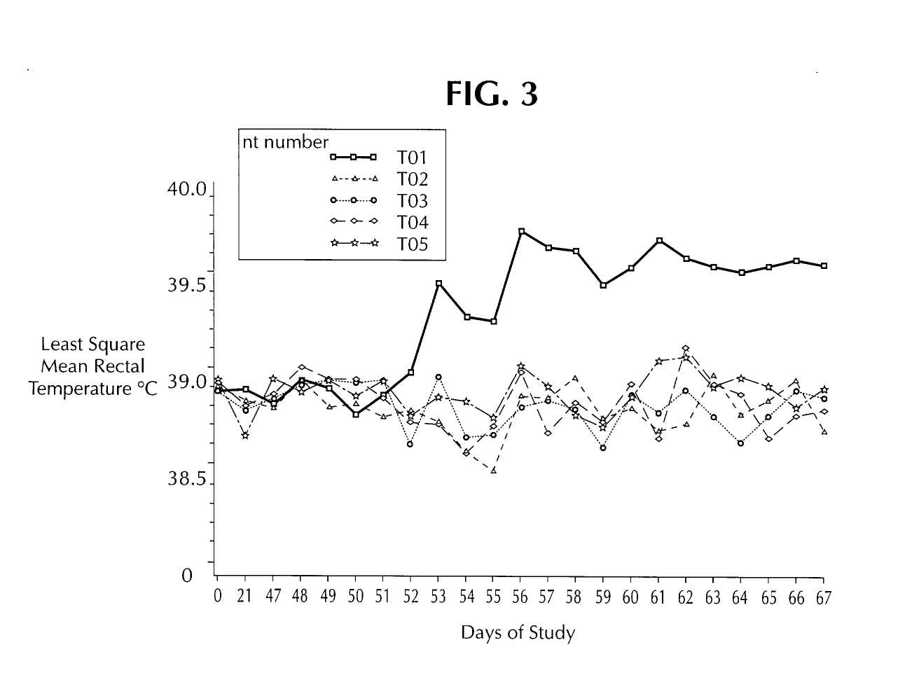

Rectal temperatures were measured each morning immediately prior to challenge (day 47) and for 20 days following experimental M. bovis challenge. Results are summarized in FIG. 3. Calves administered two doses of the M. bovis vaccines (Treatment Groups 2, 3, 4 and 5) had lower mean body temperatures on days 52 through 67 when compared to the placebo vaccinated animals (Treatment Group 1).

M. bovis specific serum antibody responses (IgG) are summarized in Table 3. Serum samples with mean percentage optical density (OD) values >0.8080% of the positive control serum were considered positive for M. bovis. All calves were M. bovis negative prior to vaccination. Calves that received the experimental M. bovis bacterins (Treatment Groups 2, 3, 4, and 5) showed an antibody response following vaccination. Animals in Treatment Group 1 (placebo vaccinated animals) were seronegative until 3 weeks following the experimental M. bovis challenge.

TABLE 3
Summary of Mycoplasma bovis Serum Antibody (IgG)
Mean Percentage of Optical Density Values to Positive
Control Serum ± Standard Deviation

| Treat- | | Prior to | | |
|---|---|---|---|---|
| ment | Pre- | Second | Prior to | 3 Weeks Post- |
| Group | Vaccination | Vaccination | Challenge | Challenge |
| 1 | 7.04 ± 13.69 | 28.14 ± 31.58 | −5.33 ± 52.24 | 183.67 ± 51.32 |
| 2 | 2.77 ± 10.47 | 79.59 ± 71.35 | 49.78 ± 34.91 | 294.75 ± 29.32 |
| 3 | 7.40 ± 13.20 | 98.21 ± | 69.77 ± 27.44 | 298.29 ± 21.13 |
| | | 102.30 | | |
| 4 | 8.34 ± 14.00 | 87.15 ± 56.79 | 65.43 ± 40.81 | 295.47 ± 26.59 |
| 5 | 5.54 ± 10.02 | 62.40 ± 72.18 | 68.31 ± 20.88 | 300.13 ± 22.91 |

All animals were necropsied at approximately 3 weeks following the experimental M. bovis challenge. Lungs were removed and evaluated grossly for characteristic lesions attributable to a M. bovis infection. Least square mean (LSM) percent lung damage scores and percent reduction of lung lesions are summarized in Table 4. Calves that were administered the experimental M. bovis bacterins (Treatment Groups 2, 3, 4, and 5) had lower LSM percent lung damage scores when compared to the placebo vaccinated animals (Treatment Group 1). These results demonstrate that two doses of the experimental M. bovis bacterins were capable of inducing protection in calves following experimental challenge.

TABLE 4
Summary of LSM Percent Lung Damage
Scores Mean Weighted Percentage

| Treatment | | |
|---|---|---|
| Group | LSM Percent Lung Damage | Percent Reduction |
| 1 | 6.5 | — |
| 2 | 0.7 | 89.23 |
| 3 | 0.9 | 86.15 |
| 4 | 2.8 | 56.92 |
| 5 | 2.9 | 55.38 |

Each lung was lavaged with 50 ml of PBS. Results of the presence of M. bovis in bronchial lavage samples by PCR approximately twenty-one days following the experimental M. bovis challenge are summarized in Table 5. Calves that were administered the experimental M. bovis bacterins (Treatment Groups 2, 3, 4, and 5) had a reduced incidence M. bovis in lung lavage samples by PCR when compared to the placebo vaccinated calves (Treatment Group 1).

TABLE 5
Summary of the Presence of Mycoplasma bovis by
PCR in Lung Lavage Fluid

| | Number of Animals | Percent |
|---|---|---|
| Treatment Group | M. bovis Positive | Positive |
| 1 | 14/16 | 87.5 |
| 2 | 0/17 | 0 |
| 3 | 4/12 | 25.0 |
| 4 | 2/15 | 11.8 |
| 5 | 1/16 | 5.9 |

In conclusion, calves receiving the experimental M. bovis bacterins (Treatment Groups 2, 3, 4, and 5) developed less lung lesions, had reduced rectal temperatures, increased average daily weight gain, and a reduced incidence of M. bovis in lung lavage samples when compared to the placebo administered animals (Treatment Group 1). The results showed that two doses of the M. bovis bacterins were capable of inducing a serological response and protection from a M. bovis experimental challenge. In addition, the results revealed a vaccine containing a single M. bovis strain is capable of protecting calves following experimental challenge with a distinctly different strain.

1. A vaccine formulation for immunization of an animal comprising an immunologically effective amount of an inactivated, whole or partial Mycoplasma bovis cell and a pharmaceutically acceptable carrier.

2. The vaccine formulation according to claim 1, further comprising an adjuvant.

3. The vaccine formulation according to claim 2 wherein said adjuvant is selected from the group consisting of Quil A or GPI-0100, saponin, cholesterol, DDA, aluminum gel, carbopol, Amphigen, Alhydrogel, oil in water, water in oil, cytokines, and combinations of adjuvants.

4. The vaccine formulation according to claim 1, further comprising an inactivating agent.

5. The vaccine formulation according to claim 4, wherein said inactivating agent is binary ethyleneimine (BEI).

6. The vaccine formulation according to claim 1, wherein the animal is a bovine.

7. The vaccine formulation according to claim 1, wherein the animal is a calf

8. The vaccine formulation of claim 1, wherein the effective amount of the M. bovis vaccine contains from about 1×10⁶ to about 5×10¹⁰ colony forming units (CFU) per dose.

9. The vaccine formulation according to claim 8 wherein the effective amount of the M. bovis vaccine contains from about 1×10⁸ to about 5×10¹⁰ colony forming units (CFU) per dose.

10. The vaccine formulation according to claim 9 wherein the effective amount of the M. bovis vaccine contains from about 5×10⁸ to about 5×10¹⁰ colony forming units (CFU) per dose.

11. The vaccine formulation according to claim 1 wherein the Mycoplasma bovis vaccine further comprises a viral or bacterial respiratory, enteric, or reproductive pathogen antigens.

12. The vaccine formulation according to claim 11 wherein said respiratory antigens are selected from the group consisting of bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica.

13. A method of treating or preventing a disease or disorder in an animal caused by infection with Mycoplasma bovis, comprising administering to the animal, an effective amount of a Mycoplasma bovis vaccine.

14. The method according to claim 13 wherein the Mycoplasma bovis vaccine reduces weight loss.

15. The method according to claim 13 wherein the Mycoplasma bovis vaccine reduces the incidence of other viral and baterial pathogens.

16. The method according to claim 13 wherein the Mycoplasma bovis vaccine reduces the incidence of mastitis.

17. The method according to claim 13 wherein the Mycoplasma bovis vaccine reduces lung lesions.

18. The method according to claim 13 wherein the Mycoplasma bovis vaccine reduces respiratory infections.

19. The method according to claim 13 wherein the animal is a bovine.

20. The method according to claim 13 wherein the animal is a calf.

21. The method according to claim 13 wherein the Mycoplasma bovis vaccine formulation is an inactivated, whole or partial Mycoplasma bovis cell preparation.

22. The method according to claim 13, wherein the effective amount of the M. bovis vaccine contains from about 1×10⁶ to about 5×10¹⁰ colony forming units (CFU) per dose.

23. The method according to claim 22 wherein the effective amount of the M. bovis vaccine contains from about 1×10⁸ to about 5×10¹⁰ colony forming units (CFU) per dose.

24. The method according to claim 23 wherein the effective amount of the M. bovis vaccine contains from about 5×10 8 to about 5×10¹⁰ colony forming units (CFU) per dose.

25. The method according to claim 13 wherein the amount of said vaccine administered is from about 0.5 to about 5.0 ml.

26. The method according to claim 13 wherein the amount of said vaccine administered is from about 1.5 ml to about 2.5 ml.

27. The method according to claim 13 wherein the amount of said vaccine administered is about 2 ml.

28. The method according to claim 27 wherein about two milliliters of the vaccine are administered twice to the calf.

29. The method according to claim 28 wherein the two administrations of the vaccine occur first at about three weeks and then at about six weeks after the birth of the calf.

30. The method according to claim 13 wherein said Mycoplasma bovis cell preparation is administered subcutaneously.

31. The method according to claim 13 wherein said Mycoplasma bovis cell preparation is administered intranasally.

32. The method according to claim 13 wherein said Mycoplasma bovis cell preparation is administered intramuscularly.

33. The method according to claim 13 wherein the Mycoplasma bovis vaccine further comprises an adjuvant.

34. The method according to claim 13 wherein the Mycoplasma bovis vaccine further comprises an inactivating agent.

35. The method according to claim 34 wherein said inactivating agent is binary ethyleneimine (BEI).

36. The method according to claim 33 wherein the adjuvant is selected from the group consisting of Quil A or GPI-0100, saponin, cholesterol, DDA, aluminum gel, carbopol, Amphigen, Alhydrogel, oil in water, water in oil, cytokines, or combinations of adjuvants.

37. The method according to claim 13 wherein the Mycoplasma bovis vaccine further comprises a pharmaceutically acceptable carrier.

38. The method according to claim 13 wherein the Mycoplasma bovis vaccine further comprises a viral or bacterial respiratory enteric, or reproductive pathogen antigens.

39. The method according to claim 38 wherein said respiratory antigens are selected from the group consisting of bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica.

40. A method of preparing a Mycoplasma bovis vaccine comprising growing a isolate of Mycoplasma bovis in culture in a suitable medium; treating the Mycoplasma bovis with binary etheleneimine to inactivate the Mycoplasma bovis; and admixing the inactivated Mycoplasma bovis with a suitable pharmaceutically acceptable carrier.

41. The method according to claim 40 wherein the Mycoplasma bovis vaccine reduces shedding and transmission of Mycoplasma bovis.

42. The method according to claim 40 wherein the Mycoplasma bovis vaccine induces a immune response.

43. A kit comprising in at least one container a Mycoplasma bovis bacterin and an adjuvant.

44. The kit according to claim 43 wherein said adjuvant is selected from the group consisting of Quil A, saponin, cholesterol, aluminum gel, DDA, carbopol, Amphigen, Alhydrogel, oil in water, water in oil, cytokines, or combinations of adjuvants.

45. The kit according to claim 43 further comprising in at least an antigen selected from the group consisting of bovine herpesvirus type 1 (BHV-1), bovine viral diarrhea virus (BVDV), bovine respiratory syncitial virus (BRSV), parainfluenza virus (PI3), Pasteurella multocida, Haemophilus somnus, Mycoplasma mycoides, Mycoplasma agalactiae, Mycoplasma californicum, Mycoplasma bovirhinis, Mycoplasma dispar, Mycoplasma canis, and Manheimia haemolytica.

46. A bacterin comprising an inactivated Mycoplasma bovis isolate in an amount of about 5×10⁸ colony forming units per dose of bacterin, in a pharmaceutically acceptable carrier.

47. The bacterin according to claim 46, further comprising an adjuvant.

48. The bacterin according to claim 46, wherein said adjuvant is selected from the group consisting of Quil A or GPI-0100, saponin, cholesterol, DDA, aluminum gel, carbopol, Amphigen, Alhydrogel, oil in water, water in oil, cytokines, or combinations of adjuvants.

49. The bacterin according to claim 46, further comprising an inactivating agent.

50. The bacterin according to claim 49 wherein said inactivating agent is binary ethyleneimine (BEI).

51. A vaccine formulation for immunization of an animal comprising an immunologically effective amount of an inactivated, whole or partial Mycoplasma bovis cell and an adjuvant comprising Quil A, cholesterol and Amphigen.

52. A method of treating or preventing a disease or disorder in an animal caused by infection with Mycoplasma bovis and at least one other heterologous strain, comprising administering to the animal, an effective amount of a Mycoplasma bovis vaccine.

53. A vaccine formulation for immunization of an animal comprising an immunologically effective amount of an inactivated, whole or partial Mycoplasma bovis cell and OneShot™ (M.haemolytica), or Bovishield™ (BRSV/BVD-1/BVD-2/BHV-1/PI3, or H. somunus or P. Multocida, or any combination thereof.

54. A vaccine formulation for immunization of an animal comprising an immunologically effective amount of an inactivated, whole or partial Mycoplasma bovis cell, wherein the Mycoplasma bovis is the strain designated as ATCC PTA-3685.

55. A method for treating or preventing a disease or disorder having the clinical manifestations of pneumonia of calves, which is often accompanied by arthritis, also known as pneumonia-arthritis syndrome by administering to the calves an immunologically amount of the vaccine of claim 1.
